(19) United States
(12) Patent Application Publication
(10) Pub. No.: US 2007/0148038 A1
(43) Pub. Date: Jun. 28, 2007

(54) Plug-In Oxygenator

(76) Inventor: Shaam P. Sundhar, Princeton, NJ (US)

Correspondence Address: PATWRITE LLC, 408 W. MAIN ST., MARSHALLTOWN, IA, 50158-5759, US

(21) Appl. No.: 11/380,041

(22) Filed: Apr. 25, 2006

Related U.S. Application Data
(60) Provisional application No. 60/594,630, filed on Apr. 25, 2005.

(51) Int. Cl. A61M 1/00

(52) U.S. Cl. 422/46; 454/69

(57) ABSTRACT

A plug-in oxygenator for use in a vehicle to enrich the oxygen levels of the interior space of the vehicle. The oxygenator includes a plug end that fits into the auxiliary power outlet common to most vehicles also known as a cigarette lighter socket. As the oxygenator heats up, it releases oxygen which is diffused within the vehicle. When the pellet is consumed, it is replaced and the oxygenator is ready for repeated use. A thermostat regulates the temperature of the oxygenator and an insulative layer surrounding an inner housing protects the user from burns and exposure to high temperature.

### RELATED APPLICATIONS

This application claims priority and herein incorporates by reference U.S. provisional patent application No. 60/594,630, filed Mar. 25, 2005.

### BACKGROUND OF THE INVENTION

Many people are convinced that breathing an atmosphere enriched in oxygen can help with fatigue and other symptoms in today's fast paced industrialized world. In countries such as Japan, oxygen bars have been around for years and in some areas of the United States such as California, more and more oxygen bars are opening up daily. Although the bars are often equipped with music, video and reading materials, clients using the services must go to the bar and spend valuable time in order to receive the benefit of breathing an atmosphere enriched in oxygen. Furthermore, many of these establishments scent the oxygen in order to make the experience more pleasant, but there is some evidence to suggest that breathing these vaporized scents may pose a health risk. There are many companies who manufacture and market pressurized oxygen cans. They are bulky and expensive when used on a regular basis.

Many people in the industrialized world spend time commuting to work in an automobile. The effects of heavy traffic, stress and lack of sleep and fresh air is likely the cause for many automobile accidents. The modern commute is becoming synonymous with spending longer and longer periods in your car. There is a need for an oxygenator that the user can operate effortlessly without wasting time and money going to an oxygen bar.

### SUMMARY OF THE INVENTION

A plug-in oxygenator for use in a vehicle to enrich the oxygen levels of the interior space of the vehicle. The oxygenator includes a plug end that fits into the auxiliary power outlet common to most vehicles also known as a cigarette lighter socket. As the oxygenator heats up, it releases oxygen which is diffused within the vehicle. When the pellet is consumed, it is replaced and the oxygenator is ready for repeated use. A thermostat regulates the temperature of the oxygenator and an insulative layer surrounding an inner housing protects the user from burns and exposure to high temperature.

Other features and advantages of the instant invention will become apparent from the following description of the invention which refers to the accompanying drawings.

### BRIEF DESCRIPTION OF THE DRAWINGS

FIG. 1 shows a side view of an embodiment of the invention.

FIG. 2 illustrates the invention in a typical application.

FIG. 3 is a top view of the cap portion of an embodiment of the invention.

FIG. 4 is a side view of another embodiment of the invention.

FIG. 5 is yet another embodiment of the invention.

FIG. 6 depicts a further embodiment of the invention.

FIG. 7 depicts yet another embodiment of the invention.

FIG. 8 diagrams another embodiment of the invention.

FIG. 9 is an enlarged view illustrating a core and oxygenating pellet according to the present invention.

### DETAILED DESCRIPTION OF THE INVENTION

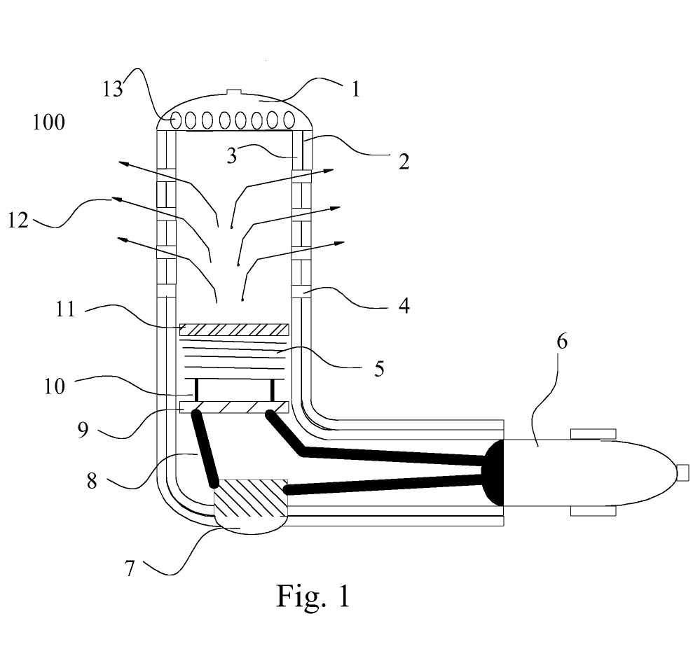

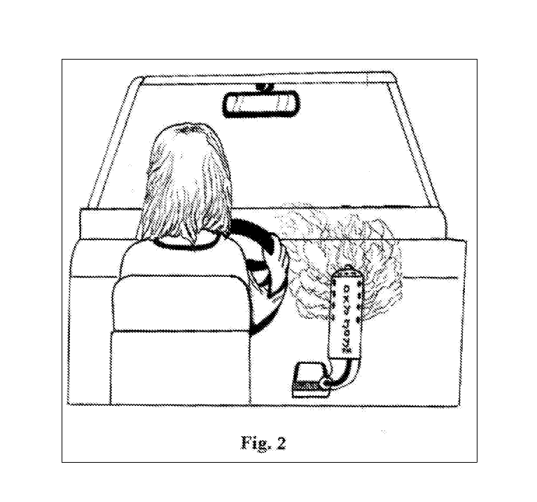

Referring to FIGS. 1, 3 and 9, a Plug-In Oxygenator (shown generally as 100), consists of a housing 3 covered with a thermally insulative layer 2. Housing 3 is made of steel, but any suitable material such as aluminum or even a high temperature plastic may be used. A resistive heating element 5 is wrapped around a thermally conductive core 11 and thermally isolated from a bottom portion of Oxygenator 100 by insulative posts 10 and a heat isolation ring 9. Heating element 5 is electrically connected to a thermostat 7 using lead wires 8 which are electrically connected to a plug end 6 which fits into the auxiliary output of a motor vehicle (shown generally in FIG. 2) also known as a cigarette lighter socket. An oxygenating pellet 50 is inserted to make thermal contact with core 11. As pellet 50 is heated, oxygen is released. Oxygen diffuses (shown generally as 12) through a plurality of exhaust ports 4 and venting ports 13 to increase the oxygen level of the motor vehicle.

Thermostat 7 regulates the temperature of core 11 and this controls the amount of oxygen released. The higher the temperature, the more oxygen is released; however, higher operating temperatures reduce the time that pellet 50 will last. Thermostat 7 operates on direct current and in an embodiment, a user selectable switch (not shown) is provided to allow the user to select the operating temperature and therefore the time that pellet 50 will last before needing replacement. In the embodiment shown, thermostat 7 maintains a preset temperature.

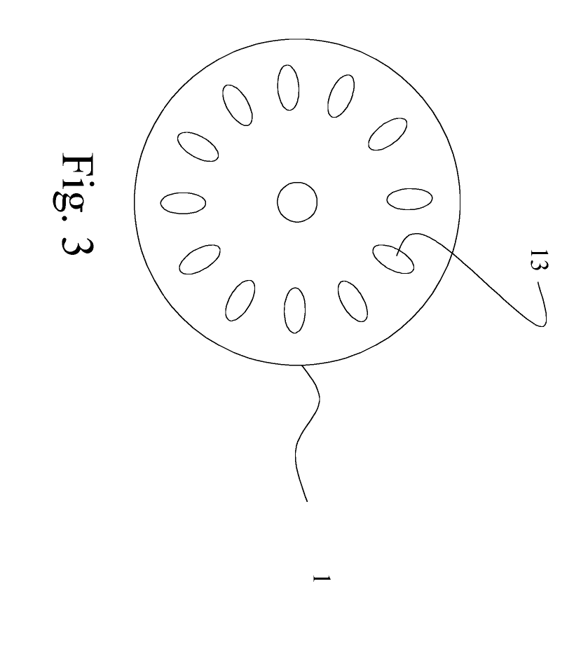

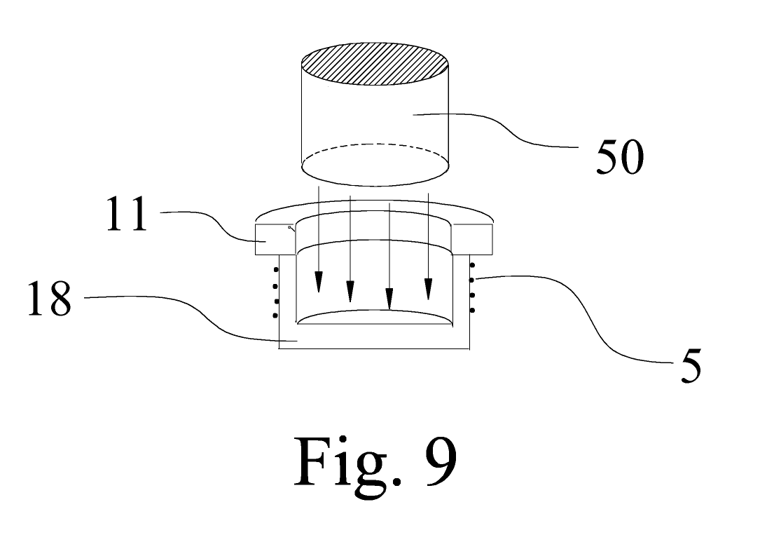

Referring now to FIGS. 3 and 9, a user removable cap 1 allows a user to place pellet 50 in core 11 and to clean out any debris left behind when pellet 50 is consumed. Cap 1 press-fits into housing 3 utilizing friction to hold cap 1 in place while in use. Cap 1 is removed after consumption of pellet 50 and oxygenator 100 is removed from the cigarette light socket (not shown) and turned upside down and gently tapped until the used ash falls free from core 11. To place oxygenator 100 back in service, a new pellet 50 is reinserted into core 11 and cap 1 is replaced, and then oxygenator 100 is plugged back in the socket (not shown). One embodiment of the instant invention has a switch (not shown) to allow a user to turn oxygenator 100 on or off without removing it from the socket (not shown). The plurality of vents 13 allow oxygen to diffuse through cap 1 and enter the vehicle air space in order to increase the oxygen level in the vehicle as described above. Pellet 50 fits within core 11 and is heated to operating temperatures to release oxygen. Pellets 50 can be made of compressed compounds as discussed below. The shape of pellets 50 is matched to core 11 geometry (for example triangular core geometry 19 and cylindrical core geometry 18) in order to ensure maximum thermal contact.

Pellets 50 are made of an oxidizing compound such as FeTiO₃. FeTiO₃ has many advantages in that it does not require a catalyst to produce the oxygen and one gram produces up to ten liters of O₂. Any ash left behind is non-toxic and may be safely disposed of. Additionally, FeTiO₃ can be formed into thin strips to increase the reactive area making O₂ production more efficient. Other compounds may be used to generate oxygen as is known in the art, such as Potassium Chloride or Potassium superoxide. Lithium perchlorate is used in the space program and is sometimes referred to as oxygen candles. There are three main methods of generating oxygen and the instant invention may be modified to use other known methods of creating oxygen. In addition to heating of compounds such as FeTiO₃ as described above, oxygen can be released through the electrolysis of water and by the use of selective membranes used to extract O₂ from ambient air. Additionally, H₂O₂ can also be used conjunction with Potassium Permanganate to generate oxygen. Although the embodiments depicted in this application utilize oxygenating pellets 50, other methods of making oxygen may be used.

Thermally insulative layer 2 is a high temperature plastic that serves to thermally isolate the hot housing 3 from contact by a user. Temperatures inside core 11 can reach temperatures in excess of 200 degrees Celsius. Alternatively, insulative layer 2 may be separated from housing 3 by a trapped air layer (not shown) providing even more insulative protection. Insulative layer 2 may also be made of a suitable insulative material such as ceramic or glass.

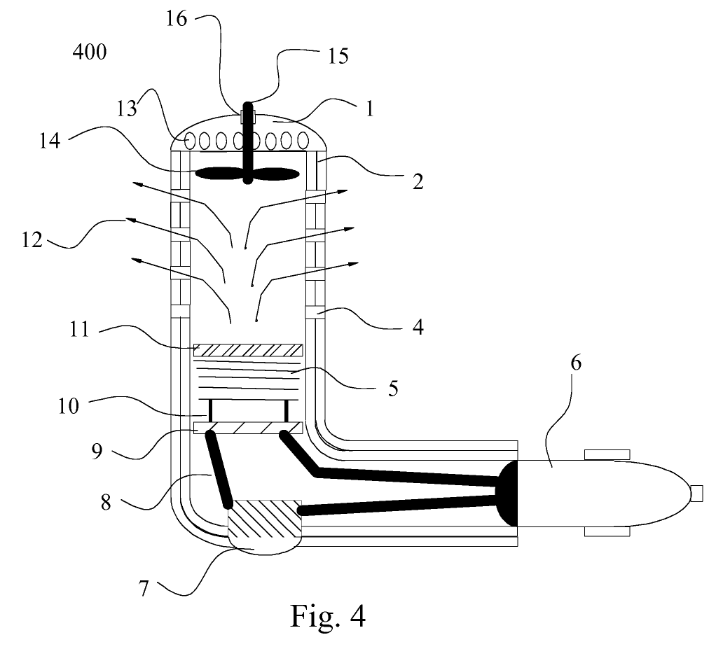

With respect to FIGS. 4-7, only those features unique to the embodiment will be discussed. Note that like reference designators on different figures refer to like elements.

FIG. 4 illustrates an embodiment of the instant invention (shown generally as 400) that includes a fan 15 rotatably connected to cap 1. A bearing sleeve 16 positions and fixes fan 15 in place. Heat rising from core 11 spins fan 15 improving the distribution of the oxygen being produced. Other fan shapes would be suitable such as a turbine type or multi-bladed design.

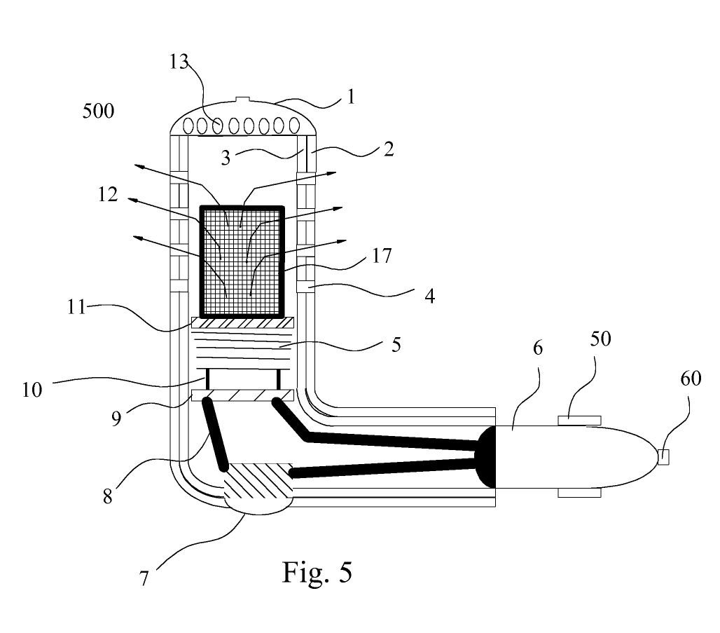

Referring now to FIG. 5, a plug-in oxygenator (shown generally as 500) includes a screen chamber 17 that traps and contains pellet 50 and any remaining ash. Oxygen flows freely through screen 17. Screen 17 is made of a metal mesh that is heat resistant and capable of withstanding temperatures in excess of 200 degrees Celsius. Alternatively, a high temperature plastic or other suitable material may be used for screen 17.

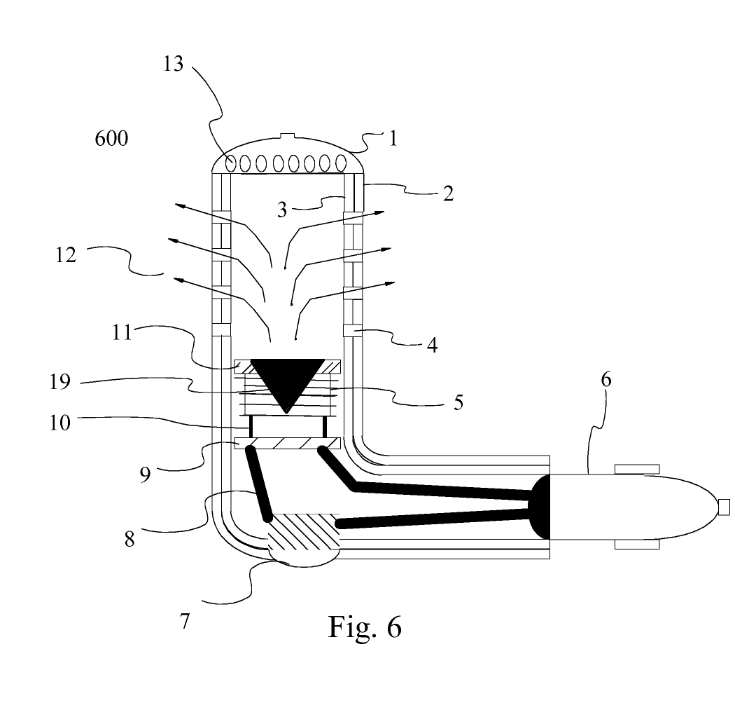

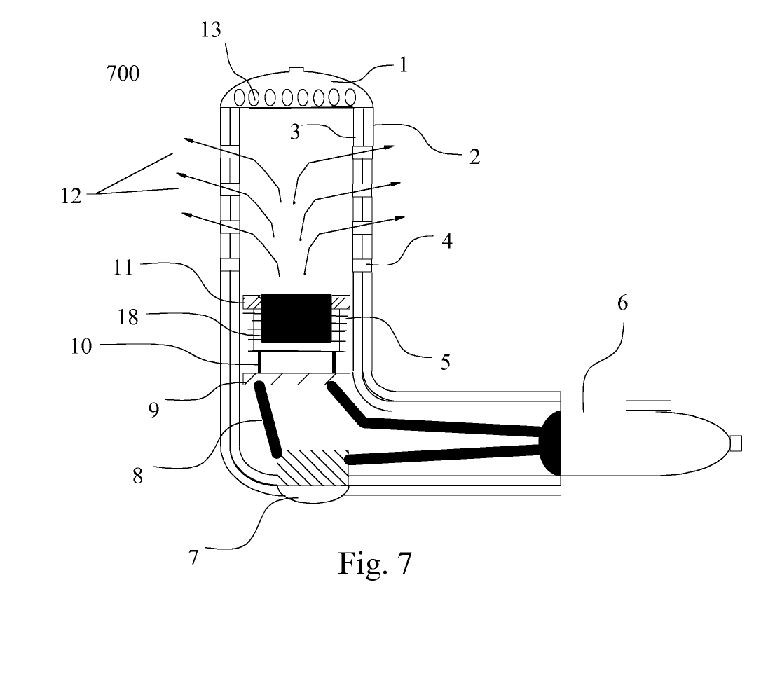

Now referring to FIG. 6 and 7, a plug-in oxygenator (shown generally as 600 and 700 respectively) features a prismatic core geometry 19 (FIG. 6) and a cylindrical core geometry 18 (FIG. 7) to help hold and position pellet 50 within core 11. Both shapes automatically stabilize pellet 50 and aid a user when inserting pellet 50 within core 11.

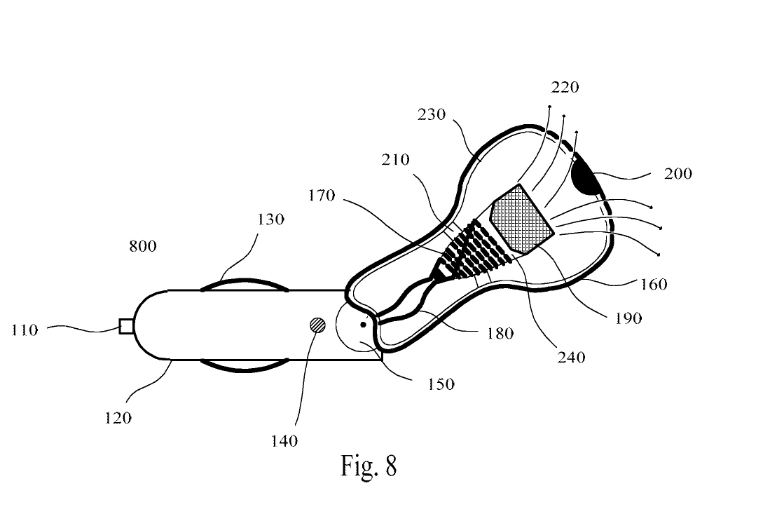

FIG. 8 depicts a variable geometry plug-in oxygenator (shown generally as 800) having a housing 230 surrounded by a thermally insulative layer 160. Housing 230 is pivotally attached to an auxiliary plug 120 for insertion into a cigarette lighter socket (not shown). Plug 120 includes a contact stud 110 and two spring contacts 130 to ensure electrical contact with the socket (not shown) as is known in the art. A hinge 150 allows housing 230 to adjust for different vehicles to accommodate different mounting geometries among automobile designs. Hinge 150 allows housing to rotate between a essentially horizontal to vertical position. A handle 200 allows a user to easily adjust the angle of plug-in oxygenator 800. Wires 180 are movably connected to plug 120 utilizing a sliding electrical contact (not shown) as is known in the art; thereby maintaining electrical contact throughout the pivot. A thermally conductive core 240 is centrally mounted within a space bounded by housing 230 and supported by thermally insulative posts 210. A resistive heating element 170 surrounds core 240 to heat core 240 to over 200 degrees Celsius. A oxygenating pellet 50 (FIG. 9) fits within core 240 and is surrounded by screen chamber 190 to catch ash. In operation, core 240 is heated and oxygen is released and diffused through venting ports (shown generally as 220) to increase the oxygen levels in the vehicle. In the embodiment shown, an LED 140 is mounted within plug 120 to indicate a power on/off status. Additionally, in an alternative embodiment (not shown) a power on/off switch allows a user to turn the unit on and off.

Although the instant invention has been described in relation to particular embodiments thereof, many other variations and modifications and other uses will become apparent to those skilled in the art.

What is claimed is:

1. An automobile plug-in oxygenator comprising:
a housing;
at least one exhaust port disposed through said housing;
a heating element disposed within said housing;
a thermally insulative layer substantially surrounding said housing;
a thermally conductive core in thermal contact with said heating element; and
a plug end distally connected to an end of said housing wherein said plug end fits a cigarette lighter/auxiliary power output of said automobile wherein said heating element is electrically energized when plugged in therein.

2. The automobile plug in oxygenator according to claim 1 further comprising a thermostat electrically connected to said heating element.

3. The automobile plug in oxygenator according to claim 2 further comprising a chemical oxidizing pellet shaped to mechanically fit within said thermally conductive core.

4. The automobile plug in oxygenator according to claim 3 wherein said chemical oxidizing pellet is Iron Titanium Oxide (FeTiO₃).

5. The automobile plug in oxygenator according to claim 3 wherein said chemical oxidizing pellet is Potassium Chloride (KCl).

6. The automobile plug in oxygenator according to claim 3 wherein said chemical oxidizing pellet is Potassium Superoxide (KO₂).

7. The automobile plug in oxygenator according to claim 3 wherein said chemical pellet is Lithium Perchlorate (LiClO₄).

8. The automobile plug in oxygenator according to claim 2 further comprising an end cap removeably attached to a free end of said housing.

9. The automobile plug in oxygenator according to claim 2 wherein said thermostat is user adjustable wherein oxygen output is controlled in response to user adjustment.

10. The automobile plug in oxygenator according to claim 9 further comprising a hinge portion disposed between said plug end and said heating element wherein said housing orientation is adjustable between zero degrees and ninety degrees with respect to said plug end.

11. The automobile plug in oxygenator according to claim 8 further comprising:
a fan disposed within said housing; and
a bearing sleeve centrally disposed on said end cap rotatably securing said fan within said housing.

12. The automobile plug in oxygenator according to claim 2 further comprising a heat isolation ring disposed between said thermally conductive core and said thermostat.

13. The automobile plug in oxygenator according to claim 2 further comprising a screen chamber removeably disposed on an upper surface of said thermally conductive core.

14. The automobile plug in oxygenator according to claim 12 wherein said thermally conductive core has a geometric interior cavity.

15. The automobile plug in oxygenator according to claim 14 wherein said geometric interior cavity is cone shaped.

16. The automobile plug in oxygenator according to claim 14 wherein said geometric interior cavity is cylinder shaped.

17. The automobile plug in oxygenator according to claim 2 further comprising a power on indicator light wherein when said heating element is energized said indicator light is also energized.

18. The automobile plug in oxygenator according to claim 2 further comprising an on-off switch disposed within said housing wherein a user can turn said heating element on and off.

19. A plug in oxygenator for use in a motor vehicle comprising:
a substantially tubular housing;
a thermally insulative layer substantially enclosing said housing;
a plurality of exhaust ports annularly orientated through said housing and said thermally insulative layer wherein gaseous exchange occurs when in use;
a heating element and core disposed within a lower portion of said housing;
a plug end distally disposed to engage with said motor vehicle's cigarette lighter/auxiliary power output; and
said heating element and core being electrically connected to said plug end.

20. A plug in oxygenator for use in a motor vehicle comprising:
a housing;
a plurality of exhaust ports disposed through said housing;
a thermally insulative layer substantially covering an outer surface of said housing;
a plug end distally disposed on an end of said housing;
a heating means for heating a chemical substance to a preselected temperature; and
connection means for electrically connecting said heating means to said plug end.
